(19) United States
(12) Patent Application Publication
(10) Pub. No.: US 2022/0000130 A1
(43) Pub. Date: Jan. 6, 2022

(54) METHODS FOR PASTEURIZING AND/OR STERILIZING PARTICULATE GOODS

(71) Applicant: BÜHLER AG, Uzwil (CH)

(72) Inventors: Nicolas MENESES, Gossau (CH); Martin HERSCHE, St. Gallen (CH); Alasdair CURRIE, London (GB); Niklaus SCHÖNENBERGER, Herisau (CH); Thomas SCHEIWILLER, Zuzwil (CH)

(21) Appl. No.: 17/475,467

(22) Filed: Sep. 15, 2021

Related U.S. Application Data
(63) Continuation of application No. 16/326,808, filed on Feb. 20, 2019.

(30) Foreign Application Priority Data
Aug. 20, 2016 (EP) 16185056.5

(51) Int. Cl. A23B 9/06; A23L 3/26; A61L 2/08; B65B 37/04; B65B 55/16

(52) U.S. Cl. CPC A23B 9/06; A23L 3/263; B65B 55/16; B65B 37/04; A61L 2/087

(57) ABSTRACT

Methods for pasteurizing and/or sterilizing particulate goods (1), containing the following steps: a) producing an electron beam (5), b) pasteurizing and/or sterilizing the goods (1) by the electron beam (5) in a treatment zone (3), wherein the electrons of the electron beam (5) have an energy that lies in the range of 80 keV to 300 keV, preferably from 140 keV to 280 keV, and more preferably from 180 keV to 260 keV, the goods (1) are exposed to the electron beam (5) for a treatment time which lies in the range from 5 ms to 25 ms, and the electron beam (5) has a mean electron current density in the treatment zone (3) which lies in the range of 10¹⁵ s⁻¹·cm⁻² to 2.77·10¹⁵ s⁻¹·cm⁻².

The present invention concerns methods for pasteurizing and/or sterilizing particulate goods by means of an electron beam.

Particulate goods are defined here and in the following as goods consisting inter alia of grains and/or flakes, whereby the particles can have a spherical, plate-shaped or angular shape. They may also be ground particles. Pasteurization and/or sterilization, for example, can kill or render harmless at least the majority of micro-organisms. In particular, a reduction of harmful microorganisms by at least five orders of magnitude can be achieved.

A device for pasteurizing and/or sterilizing particulate goods is known for example from EP 1 080 623 B1. This device contains vibrating conveyors with which seed can be separated into a transparent curtain. This curtain is then guided through an electron field generated by an electron accelerator which can, for example, sterilize the seed.

Another device known from the U.S. Pat. No. 5,801,387 A is used for pasteurizing and/or sterilizing particulate goods. In the device according to that invention, a particle-shaped good is dosed into a horizontal air stream with a vibration conveyor and then exposed to an electron beam. A vacuum pump and a filter are then used to classify the goods.

Furthermore, the DE 10 2012 209 434 A1 reveals a device that separates and rotates a free-flowing product with the aid of a vibration conveyor and a rotating brush roller. The particles then pass in free fall through an electron field.

EP 0 513 135 B1 discloses a device with which seed is introduced into a vertical chute by means of rotary valves, where it is subjected in vertical fall to electron beams.

Another device known from EP 0 705 531 B1 is a dosing device which introduces the seed into a process chamber by means of an unspecified dosing device, in which it falls vertically through an electron beam.

The device disclosed in U.S. Pat. No. 6,486,481 BI contains a vibrating table on which a polymeric good is moved and exposed to an electron beam. However, this is not done for pasteurisation or sterilization purposes, but to reduce the molecular weight of the polymeric good.

It is an aim of the present invention to overcome the disadvantages known from the prior art. In particular, methods are to be provided with which particulate goods can be pasteurised and/or sterilised effectively, reliably and as simply, quickly and inexpensively as possible.

These and other tasks are solved by the method according to the present invention of pasteurizing and/or sterilizing particulate goods. It comprises the following steps:
a) Generating an electron beam,
b) Pasteurisation and/or sterilisation of the good using the electron beam in a treatment zone.

According to the invention, the electrons of the electron beam have an energy in the range from 80 keV to 300 keV, preferably from 140 keV to 280 keV, more preferably from 180 keV to 260 keV. Lower electron energies would not produce sufficient pasteurization and/or sterilization. Higher electron energies could not achieve significantly higher degrees of pasteurization and/or sterilization.

Furthermore, according to the invention, the electron current density in the treatment zone is in the range of 10¹⁵ s⁻¹*cm ⁻² to 2,77·10¹⁵ s⁻¹*cm⁻². In this range, sufficient pasteurization and/or sterilization is achieved.

According to the invention, the good is also exposed to the electron beam for a treatment time in the range of 5 ms to 25 ms. A certain minimum treatment time is required for sufficient pasteurisation and/or sterilisation. Too long treatment times have not shown a significantly increased degree of pasteurisation and/or sterilisation.

The product can be a foodstuff such as cereals such as soya, breakfast cereals, snacks, nuts such as dried coconuts, almonds, peanut butter, cocoa beans, chocolate, chocolate liquid, chocolate powder, chocolate chips, cocoa products, pulses, coffee, seeds such as pumpkin seeds, spices (such as turmeric, particularly in slices), tea mixtures, dried fruit, pistachios, dry protein products, bakery products, sugar, potato products, pasta, baby food, dried egg products, soya products such as soya beans, thickeners, yeasts, yeast extracts, gelatine or enzymes.

Alternatively, the product may also be a pet food, such as pellets, feed for ruminants, poultry, aquatic animals (in particular fish) or pets, or compound feed.

It is, however, also conceivable and lies within the scope of the invention that the good is, for example, a plastic such as PET, for example in the form of flakes or pellets.

The electron beam is advantageously used to expose the good to a radiation dose in the range from 1 kGy to 45 kGy, preferably from 8 kGy to 30 kGy, especially preferred from 10 kGy to 16 kGy.

It is advantageous to separate the good before the treatment in step b). This separation ensures that each individual grain of the good is captured by the electron beam and thus pasteurised and/or sterilised. Separation can be achieved, for example, with the aid of vibrating surfaces which are excited to vibrate and which optionally have one or more channels. Alternatively or additionally a separation can be achieved by a sliding surface on which the good slides down.

Furthermore, it is advantageous that the good falls freely through the treatment zone. The good is called “free-falling” if the trajectories of the individual particles of the good are determined solely by their velocity, the force of gravity acting on them and, if applicable, a process gas surrounding the good. In particular, the particles of the good do not slide on a surface through the treatment zone. In free fall, the speed is independent of the throughput, so that through-puts in the range of 100 kg/h to 1000 kg/h can be achieved at the same speed.

For many goods, in particular for a large number of spices, it has proven to be advantageous if the goods move through the treatment zone at a speed ranging from 1 m/s to 5 m/s, preferably from 2 m/s to 4 m/s, particularly preferably from 2 m/s to 3 m/s. The speed of the goods is determined by the speed of the spices. The higher the speed of the goods, the higher the achievable throughput. On the other hand, the speeds must not be too high so that the goods remains in the electron beam long enough to be pasteurized and/or sterilized.

In the following, the invention is explained in more detail by way of specific embodiments and drawings.

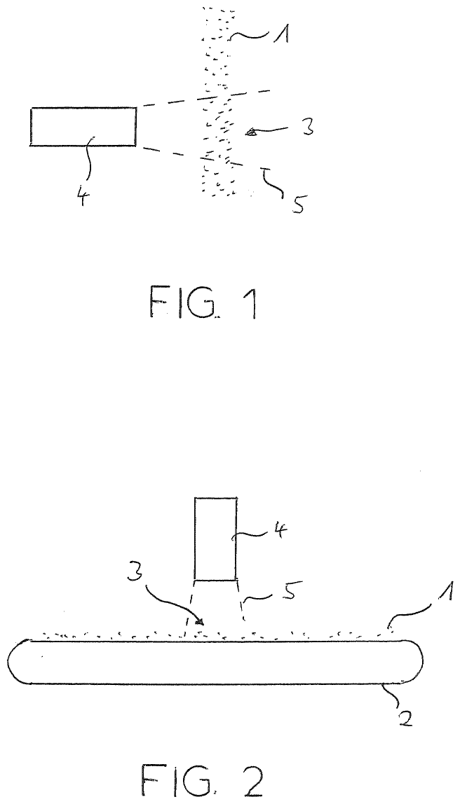

FIG. 1: a schematic representation of a first method according to the invention;

FIG. 2: a schematic representation of a second method according to the invention.

In the first embodiment schematically shown in FIG. 1, a particulate, separated good 1, such as a spice, pistachios or almonds, falls freely through a treatment zone 3 at an in-creasing speed in the range from 1 m/s to 5 m/s. There it is pasteurized and/or sterilized by means of an electron beam generated by an electron source 4. The electron beam contains electrons of an energy in the range 80 keV to 300 keV and has an average electron current density in the treatment zone 3 in the range of 10¹⁵ s⁻¹·cm⁻² to 2,77·10¹⁵ s⁻¹·cm⁻². The good 1 is subjected to this treatment for a treatment time in the range of 5 ms to 25 ms, whereby it is exposed to a radiation dose in the range 1 kGy to 45 kGy.

FIG. 2 schematically shows a second embodiment. A separated particulate good 1 is dosed onto a conveyor belt 2. The conveyor belt 2 transports the good 1 in a treatment zone 3 under an electron source 4. In treatment zone 3, the electron source 4 generates an electron beam with electrons of an energy in the range from 80 keV to 300 keV and an average electron current density in the range from 10¹⁵ s⁻¹·cm⁻² to 2,77·10¹⁵ s⁻¹·cm⁻². The good 1 is subjected to this treatment for a treatment time in the range from 5 ms to 25 ms, whereby it is exposed to a radiation dose in the range from 1 kGy to 45 kGy.

With these methods, the particulate good 1 can be pasteurized and/or sterilized effectively and reliably, but still as simply, quickly and inexpensively as possible.

1-6. (canceled)

7. A method for pasteurizing and/or sterilizing particulate material, comprising the following steps:
a) generating an electron beam,
b) pasteurizing and/or sterilizing the particulate material by means of the electron beam in a treatment zone,
wherein:
the electrons of the electron beam have an energy ranging from 80 keV to 300 keV,
the electron beam in the treatment zone has an average electron current density which is in the range from 1×10¹⁵ s⁻¹·cm⁻² to 2.77×10¹⁵ s⁻¹·cm⁻², and
the particulate material is exposed to the electron beam for a treatment time in the range from 5 ms to 25 ms.

8. The method as claimed in claim 7, wherein the electrons of the electron beam have an energy ranging from 140 keV to 280 keV.

9. The method as claimed in claim 7, wherein the electrons of the electron beam have an energy ranging from 180 keV to 260 keV.

10. The method according to claim 7, wherein the particulate material is exposed by the electron beam to a radiation dose which lies in the range from 1 kGy to 45 kGy.

11. The method according to claim 7, wherein, before step b), the particulate material is separated into individual particles.

12. The method according to claim 11, wherein the particulate material is separated into individual particles solely with a vibrating surface which is excited to vibrate and/or a sliding surface on which the particulate material slides down.

13. The method according to claim 7, wherein the particulate material falls freely through the treatment zone.

14. The method according to claim 13, wherein trajectories of the individual particles of the particulate material are determined solely by their velocity, a force of gravity acting and, if applicable, a process gas surrounding the particulate material.

15. The method according to claim 14, wherein the electron beam is produced by an electron source and the electron beam contacts the particulate material to be pasteurized and/or sterilized.

16. The method according to claim 7, wherein the particulate material moves through the treatment zone at a speed which is in the range from 1 m/s to 5 m/s.

17. The method according to claim 7, wherein the particulate material is foodstuff.

18. The method according to claim 17, wherein the foodstuff is selected from the group consisting of cereals, snacks, nuts, almonds, peanut butter, cocoa beans, chocolate, chocolate powder, chocolate chips, cocoa products, pulses, coffee, seeds, spices, tea mixtures, dried fruits, pistachios, dry protein products, bakery products, sugar, potato products, pasta, baby food, dried egg products, soya products, thickeners, yeasts, yeast extracts, gelatine and enzymes.

19. The method according to claim 7, wherein the particulate material is animal food.

20. The method according to claim 19, wherein the animal food is selected from the group consisting of pellets, feed for ruminants, poultry, aquatic animals or pets, and compound feed.

21. The method according to claim 7, wherein the particulate material is plastics.

22. The method according to claim 21, wherein the plastic is PET.
